SEQUENCE LISTING (1 sequence)

SEQ ID NO: 1 - DNA, 575 bp, Sea urchin arylsulfatase gene
gggagcagaacccctgtaagctcaggggtttttaggcgtttaattacggtcagatgagcaatttttgataccttaatttgttgtgacactggtagttaactattcattctttcttgctctcttgctttctatttctctttaaaatgcttttccaaaagaatggggggggggggctggacgctcccgaaccccgacgttccgcgggccctgacatgtaagcatctcaagaagcatatttcttgcctggctgttaatttacaaacgcataaaaaaaatataatttactaaagaatgaggaaaaatctcgggaagttatgtaatttcagcattatgtgtaaaccaccgttatggaataagaaataaaccacatttcaatttatttcccccgagcccccctccccgatcaatacgccagtgccccgccccgcccgcctcccgatctacactttgttggcggaaaaaatcgcaactgatctcccctacctttcttctttctctttcccttgctctcctcttaccctttcccttccccacccttctccacaacttgttggcgggattacctgcaaattatc

(19) United States
(12) Patent Application Publication
(10) Pub. No.: US 2005/0042204 A1
(43) Pub. Date: Feb. 24, 2005

(54) Method to protect transgenes from silencing

(75) Inventors: Masao Matsuoka, Ootsu City (JP); Koji Akasaka, Higashihiroshima City (JP)

Correspondence Address: BURNS DOANE SWECKER & MATHIS L L P, POST OFFICE BOX 1404, ALEXANDRIA, VA, 22313-1404, US

(73) Assignee: KYOTO UNIVERSITY, Kyoto (JP)

(21) Appl. No.: 10/667,359

(22) Filed: Sep. 23, 2003

(30) Foreign Application Priority Data
Mar. 20, 2003 (JP) 2003-78,202

(51) Int. Cl. A61K 48/00; C12N 15/86

(52) U.S. Cl. 424/93.2; 514/44; 435/456

(57) ABSTRACT

According to the present invention, a method to protect a transgene from silencing, wherein an insulator from sea urchin arylsulfatase gene is introduced concurrently with the transgene so as to provide a novel method for stabilization of a viral vector expression, is provided. Furthermore, according to this invention, a method for introducing a gene, wherein an insulator from sea urchin arylsulfatase gene is introduced concurrently with a transgene so as to protect the transgene from silencing, is provided. Furthermore, according to this invention, a method for production of a vector, wherein an insulator from sea urchin arylsulfatase gene is introduced into the vector so as to protect the vector from silencing, is provided. Because the viral vector containing an insulator from sea urchin arylsulfatase gene can protect a transgene from silencing, a novel useful method for gene therapy is provided.

### BACKGROUND OF THE INVENTION

1. Field of the Invention

This invention relates to a method to protect a transgene from silencing, wherein an insulator from sea urchin arylsulfatase gene is introduced concurrently with the transgene. Furthermore, this invention relates to a method for introduction of a gene, wherein an insulator from sea urchin arylsulfatase gene is introduced concurrently with a transgene so as to protect a transgene from silencing. Furthermore, this invention relates to a method for production of a vector, wherein an insulator from sea urchin arylsulfatase gene is introduced into the vector so as to protect it from silencing.

2. Description of the Prior Art

Gene therapy is a method of treating a disease by introduction of a gene and it is expected as a promising future medical technology. For example, when dysfunction of a gene caused by genetic abnormality leads to occurrence of a disease in a patient, the disease may be treated by introduction of normal gene into the patient. One method of the gene therapy is ex vivo method, in which a target gene is introduced ex vivo into cells collected from a patient, consequently re-transplanted into the patient. It was of a great social interest that gene therapy against adenosine deaminase deficiency was performed according to this method. Another method of gene therapy is direct gene introduction (in vivo method). For example, administration of a vector, incorporated with a gene of envelope protein (env) and a part of gag gene of human immunodeficiency virus (HIV), to a HIV infected patient has been attempted.

Meanwhile, most critical technique in practical application of gene therapy is the method of gene introduction. Means for gene introduction can be roughly classified into those utilizing viral vectors and those utilizing non-viral vectors. Methods using viral vectors utilizing infectious viruses are superior in efficiency of gene introduction, therefore, viral vectors are adopted more frequency in practical gene therapy. There are some types of viral vectors, and such viral vectors are chosen as appropriate. Viral vectors currently in use include retroviral vectors, adenoviral vectors, adeno-associated viral vectors, herpes simplex viral vectors and lentiviral vectors.

Because genomic DNA of a retrovirus is inserted into chromosome of a host genome, the inserted DNA can be stably inherited to the next generation. Accordingly, retroviral vectors are reliable as gene carriers, and a system of gene introduction utilizing retroviral vectors may provide a therapeutic method for permanent treatment for diseases. The representative retroviral vectors may include simple retroviruses such as murine leukemia virus. Adenoviral vectors are derived from adenovirus, which is a DNA virus, and the system for gene introduction using the adenoviral vectors is the second most frequently utilized system in the conventional gene therapy. The gene introduction using adenoviral vector is efficient and it is advantageous in that it enables introduction of genes of large size into cells, because the adenoviral vectors invade into the cells by endocytosis via receptors. Lentivirus, including HIV is a complicated retrovirus infecting macrophage and lymphocyte. The lentivirus is different from the above-mentioned retrovirus and it is advantageous in that the lentivirus can introduce a gene into non-dividing cells such as nerve cells, muscle cells, and stem cells. Moreover, the lentivirus exhibits stable expression than the retrovirus, therefore, it is considered that the lentivirus is suitable for long-term therapy of genetic diseases.

Methods with non-viral vectors are superior in safety to those with viral vectors, because they do not have danger of infection. In viral vectors, some genes required for viral reproduction are depleted and therapeutic genes are inserted into the defective site in general. Therefore, the viral vectors administrated have already lost their infectious ability, however, the viral vectors may possibly recover their proliferation ability, as the result of gene recombination with endogenous retroviruses. Accordingly, a method for gene introduction not utilizing viral vectors has been investigated. Examples of such attempts may include methods using liposome, direct injection by particle bombardment and endocytosis via receptors, but practical application of such methods have not been achieved yet.

The factors concerned with expression efficiency of a gene introduced by gene therapy with viral vectors may include the phenomenon that expression efficiency depends on the site of integration in the host genome, so-called position effect, and the phenomenon that expression efficiency of gene is decreased by epigenetic alteration, so-called silencing. The phenomenon of silencing causes suppression of gene expression in the long-term, although the gene is introduced into the host body for the purpose of therapy. Considering that the therapy for a genetic disease requires treatment throughout life, the above-mentioned silencing has been a significant barrier for achievement of successful gene therapy. The mechanism involved in this gene silencing has not been elucidated yet, however, accumulating evidences suggest that the phenomena such as DNA methylation and/or histone modifications may be involved in the gene silencing.

Chromatin insulator is a DNA sequence serving as a boundary element between differentially regulated genes. Various insulators have been found in Drosophila and vertebrates including chicken, mouse and human. These insulators are considered to have two properties in common, one is the enhancer blocking activity when placed between enhancer and promoter. An enhancer means a specific sequence that enhances transcriptional activity of a promoter, and the insulator is considered to block such action of the enhancer.

The other action of the insulator is to inhibit the position effect. The position effect means a phenomenon that transcription efficiency varies according to the position on the chromatin to which a gene was introduced. The insulator suppresses influence of surrounding transcriptional environment, which causes the position effect. Therefore, the region flanked by one-pair of insulators is isolated from environment of the host chromosome, and thereby the introduced gene can be protected from the position effect to ensure independency in gene expression. Accordingly, considering the properties of insulator as described above, efficient and stable expression of the gene may possibly be achieved by introduction of the insulator into the viral vectors.

The most investigated vertebrate insulator is cHS4 insulator, which is derived from locus control region of the chicken β-globin gene. Rivella S et al. reported that the cHS4 insulator protected transgenes from position effect when introduced into moloney-murine leukemia virus (MoMLV)-based retroviral vectors (Rivella S et al. J Virol 2000; 74: 4679-4687). However, considering practical applicability as a viral vector that delivers the gene, there is a defect that the cHS4 insulator is active in only restricted cell types. Accordingly, there has been further demand on an alternative technique to enhance availability of the viral vector.

The objective of the present invention is to provide a novel method to stabilize expression of a viral vector.

### SUMMARY OF THE INVENTION

The inventors noted and investigated on an insulator from sea urchin arylsulfatase gene. The 575 base pair-fragment derived from sea urchin arylsulfatase gene locus has typical features of an insulator such as blocing enhancer/promoter interaction and inhibiting position effect. Since ArsI (Ars insulator) works in various cell types beyond species, it has been expected that the ArsI might serve as a universal insulator working in human tissues. The base sequence of ArsI is shown in SEQ ID NO: 1 in the sequence listing.

In the previous study, ArsI has been shown to block enhancer function and suppress position effect. However, it is not known whether ArsI suppresses silencing or not. That is to say, the presence of the insulator from sea urchin arylsulfatase gene has been known, but its availability in gene therapy has not been proved because anti-silencing activity of ArsI has not been shown. Accordingly, the inventors tested for anti-silencing activity of ArsI in cell lines such as HL-60 from human leukemia. As a result, ArsI could protect the transgene from silencing, suggesting that Ars] may be effective in stabilization of viral vector expression in gene therapy. Moreover, considering that ArsI does not reduce the amount of virus prepared, ArsI is valuable in gene therapy. Furthermore, ArsI is the only effective insulator in embryonic stem cell (ES cell), which currently attract attention in the field of embryological engineering.

Accordingly, this invention provides a method to protect from silencing of a transgene, wherein an insulator from sea urchin arylsulfatase gene is introduced concurrently with the transgene. Furthermore, this invention provides a method for introduction of a gene, wherein an insulator from sea urchin arylsulfatase gene is introduced concurrently with a transgene so as to protect the transgene from silencing. Furthermore, this invention provides a method for production of a vector, wherein an insulator from sea urchin arylsulfatase gene is introduced into the vector so as to protect the vector from silencing.

These and other advantages of this invention will be apparent from a reading of the following detailed description and the drawings.

### BRIEF DESCRIPTION OF THE DRAWINGS

FIG. 1 is a schematic diagram showing the structure of the ArsI-containing viral vector.

FIG. 2 is a photograph showing detection of introduced ArsI by PCR.

FIG. 3 is a histogram of FACS analysis showing EGFP expression in HL-60 cells transduced with the ArsI-containing lentiviral vector.

FIG. 4 is a graph showing time course of anti-silencing activity represented by percentage of EGFP expression in HL-60 cells transduced with the ArsI-containing lentiviral vector.

FIG. 5 is a graph showing time course of anti-silencing activity represented by mean fluorescence intensity in HL-60 cells transduced with ArsI-containing lentiviral vector.

FIG. 6 is a histogram of FACS analysis showing EGFP expression in HL-60 cells transduced with MoMLV-based vector containing ArsI or cHS4.

FIG. 7 is a graph showing time course of anti-silencing activity represented by percentage of EGFP expression in HL-60 cells transduced with MoMLV-based vector containing ArsI or cHS4.

FIG. 8 is a graph of clonal analysis of transformed HL-60 cells.

### DETAILED DESCRIPTION OF THE INVENTION

In the following examples, the inventors investigated on the effect of an insulator from sea urchin arylsulfatase gene (ArsD) on silencing and position effect in lentiviral vectors. For the purpose of this investigation, lentiviral vectors containing ArsI in sense and anti-sense orientation in 3′-U3 region (HR/A+ and HR/A−, respectively) were constructed, respectively. Furthermore, control vector (HR/cHG) was also constructed. A fused gene (HygEGFP) comprising the hygrornycin resistance gene and the enhanced green fluorescence protein (EGFP) gene was used as the reporter gene.

Effect of ArsI on silencing was investigated using HL-60 cells. EGFP expression from HR/cHG (control vector) and HR/A+ (sense orientation vector) diminished with time, while EFGP expression from HR/A− (anti-sense orientation vector) was maintained throughout the assay period. Therefore, anti-silencing effect of ArsI was only observed in anti-sense orientation, ArsI protected lentiviral vector from silencing in an orientation-dependent manner. Furthermore, effect of ArsI on silencing was investigated in retroviral vectors containing ArsI in sense and anti-sense orientation (moloney-murine leukemia virus-based vector). As a result, anti-sense orientation-specific effect of anti-silencing was observed in the retroviral vectors as well as in the lentiviral vectors.

The inventors tested for variation of EGFP expression level to investigate the effect of ArsI on position effect. EGFP expression level of HR/cHG (control vector) varied with coefficient of variance (CV) of 39.8. Meanwhile, both HR/A+ (sense orientation vector) and HA/A− (anti-sense orientation vector) clones showed unified expression with CV of 8.3 and 23.3, respectively. These results indicated that ArsI protected lentiviral vectors from position effect regardless of the orientation, suggesting that this activity is independent of silencing protection. It has generally been considered that long-term silencing protection is likely to be a consequence of position effect protection. However, these observations indicate that different mechanisms are involved in both phenomena.

Previously, the ability to suppress position-effect and the activity to blockade enhancer effect of ArsI have been shown. However, the anti-silencing effect of ArsI has not been known. Accordingly, it is firstly found by this invention that ArsI has prominent anti-silencing effect. The use of the viral vectors, incorporated with ArsI in anti-sense orientation, may prevent decrease in expression efficiency of transgenes caused by silencing. Moreover, ArsI did not reduce the viral production. Therefore, the effect of the viral vectors introduced with ArsI is stable for a long term, and property of such viral vectors is suitable for gene therapy.

In conducting the present invention, a gene cassette, comprising a promoter placed at 5′-upstream of the exogenous gene to be introduced, may be prepared. Since an exogenous gene does not exhibit high expression efficiency in general, a promoter or an enhancer may be placed at the upstream region of the exogenous gene, for the purpose to increase the expression of the exogenous gene. For example, cytomegarovirus (CMV) promoter can be utilized for this purpose. However, the promoter enhancer to be used in this invention is not to be limited to the promoter and various promoters typically utilized in this technical field can be adopted, as appropriate. In the preferred embodiment, ArsI can be introduced in anti-sense orientation into 5′ upstream and 3′ down-stream of the gene cassette to flank the gene cassette. However, this invention is not to be limited to such embodiment. ArsI may be introduced in sense orientation into 5′ upstream and 3′ downstream, and ArsI may be introduced in either one of 5′ upstream or 3′ downstream. Therefore, such embodiments are also included within the scope of the invention, so long as ArsI exhibits anti-silencing effect.

The scope of the exogenous gene to be introduced in this invention is not to be limited to some specific embodiments, and the most adequate gene can be selected according to the purpose. In an example of the preferred embodiment of this invention, when a disease is caused by deficiency in expression of a specific gene, fundamental therapy of the disease may be achieved by introduction of the deficient gene using the viral vector containing ArsI. In this case, the causal gene of the disease may be selected as the exogenous gene to be introduced.

The gene may be introduced into living bodies, organs of the living body and cells derived from the living body using the viral vector containing ArsI. Herein living body means any animal or plant and the scope of the living body should not be particularly limited. The target gene may be introduced directly into a living body and the target gene may be introduced into organs or cells collected from the living body, and such embodiments are also included within the scope of the invention. For example, blood cells collected from the living body may be introduced with an exogenous gene using viral vectors containing ArsI, and may be provided for the purpose of gene therapy. Therefore, such method is also included within the scope of the invention. Furthermore, stem cells may be collected from respective tissue or organ, and an exogenous gene may be introduced into the cells using viral vectors containing ArsI, and thereby cells exhibiting stable expression of the exogenous gene may be constructed. Specifically, gene introduction by viral vectors containing ArsI may be achieved in blood stem cells, as well as in nervous stem cells and liver stem cells.

### EXAMPLES

(Preparation of Insulator-Containing Lentiviral Vectors)

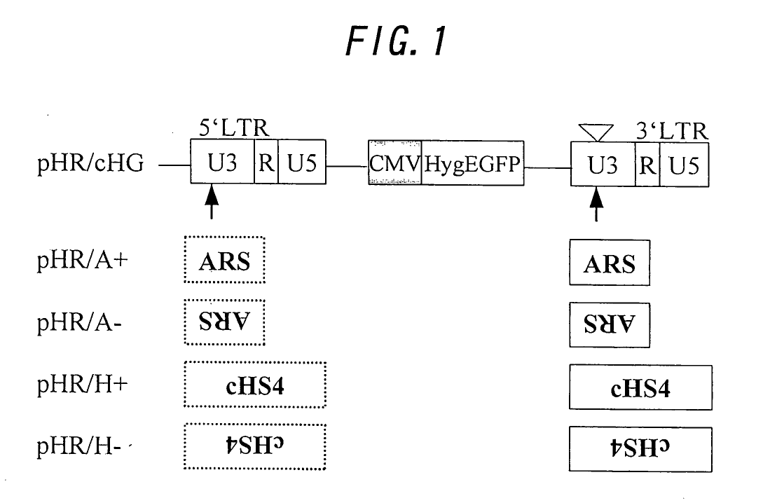

Previous studies have shown that transgenes flanked by two insulators were protected from position effects and/or silencing. Therefore, to flank the promoter/reporter cassette within the context of an HIV vector, the inventors introduced insulator fragments into the lentiviral vector, pHR, by replacing the EcoRV-EcoRV fragment of the 3′-U3 region with insulators (FIG. 1).

FIG. 1A is a schematic diagram of the lentiviral vectors. Top of FIG. 1 is a view of control vector, pHR/cHG. pHR/cHG contains HygEGFP reporter driven by cytomegalovirus immediate early promoter and 79-bp deletion in the 3′-U3 region (triangle). In middle and bottom, vectors containing ArsI in the 3′-U3 region of pHR/cHG are shown. pHR/A+ carries ArsI in sense orientation of its native genomic locus, whereas pHR/A− carries it in anti-sense orientation. Dotted boxes illustrate ArsI copied into 5′-U3 region after integration. Arrow indicates the insertion site of the insulators. pHR/H+ and pHR/H− are the vectors containing cHS4 insulator, which were constructed as well as pHR/A, and they carry cHS4 insulator in sense and anti-sense orientation, respectively.

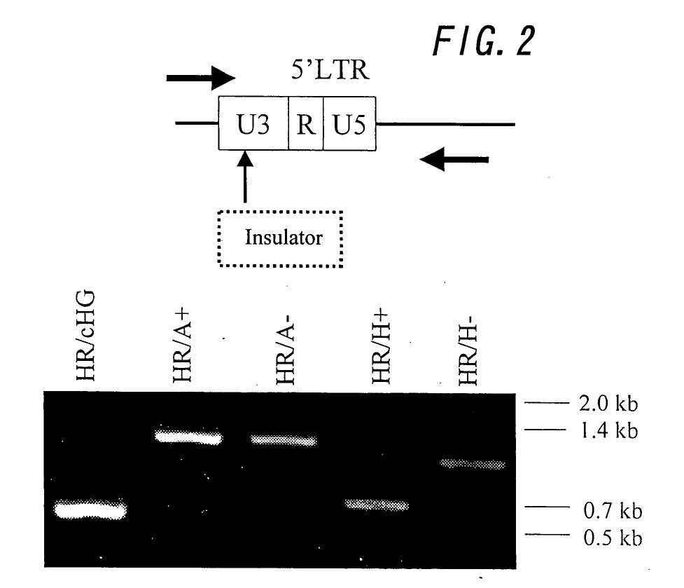

By this strategy, the inserted ArsI should be copied to 5′-long terminal repeat (LTR) during reverse transcription and thereby ArsI in the 5′-and 3′-U3 regions would flank the whole reporter cassette. This reporter cassette comprises of the cytomegalovirus (CMV) promoter and Hy&EGFP under control of the CMV promoter. Amplification of the 5′-LTR by polymerase chain reaction (PCR) shows that ArsI was successfully copied to the 5′-U3 region as expected (FIG. 2). In FIG. 2, ArsI introduced into 3′-U3 was copied to 5′-U3 during the reverse transcription. Proviral sequences from lentiviraily transduced HL-60 cells were amplified by PCR using primers specific for 5′-LTR. The expected size of the LTR without insulators was approximately 0.7 kb, whereas the size with intact ArsI was 1.3 kb.

Vector titers for HR/A+ and HR/A− were similar to the control vector, HR/cHG, indicating that ArsI does not interfere with lentiviral vector packaging and transduction (Table 1). Incorporation of cHS4 insulator into U3 region of LTR decreased the viral production, and the cHS4 sequence in 5′-LTR was partially deleted in the provirus (FIG. 2). Therefore, the inventors could not test the ability of cHS4 in context of an HIV-based vector.

TABLE 1

| Vector | Titer (TU/mL) |
|---|---|
| HR/cHG | 1.5 × 10⁵ |
| HR/A+ | 1.1 × 10⁵ |
| HR/A− | 1.1 × 10⁵ |
| HR/H+ | 3.9 × 10⁴ |
| HR/H− | 6.6 × 10⁴ |

(Effect of Ars Insulator on Lentiviral Vector Silencing.)

For silencing protection assay, the inventors introduced lentiviral vectors into HL,60 cells. The cells were transduced with lentiviral vectors, RR/cHG, HR/A+ or HR/A−, and were selected for hygromycin resistant cells. After the inventors confirmed that almost all cells were positive for expression of enhanced green fluorescence protein (EGFP), such cells were selected and cultured with hygromycin for an additional 2 weeks to eliminate untransduced cells. Thereafter, the cells were cultured without hygromycin and periodically tested for EGFP expression. Long-term silencing was observed in HL-60 cells.

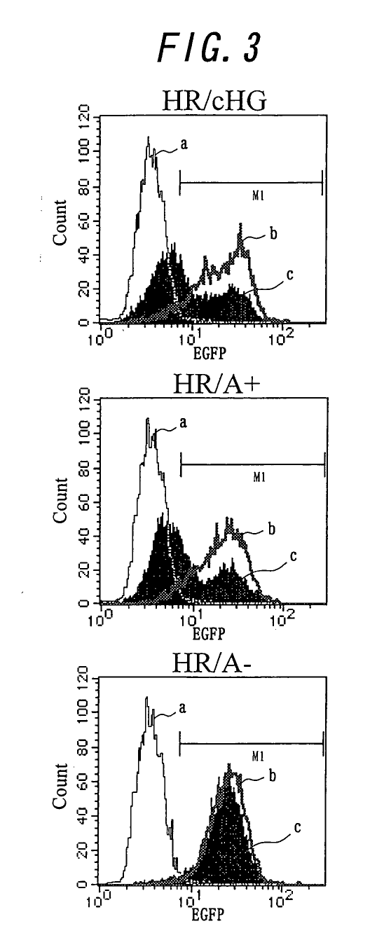

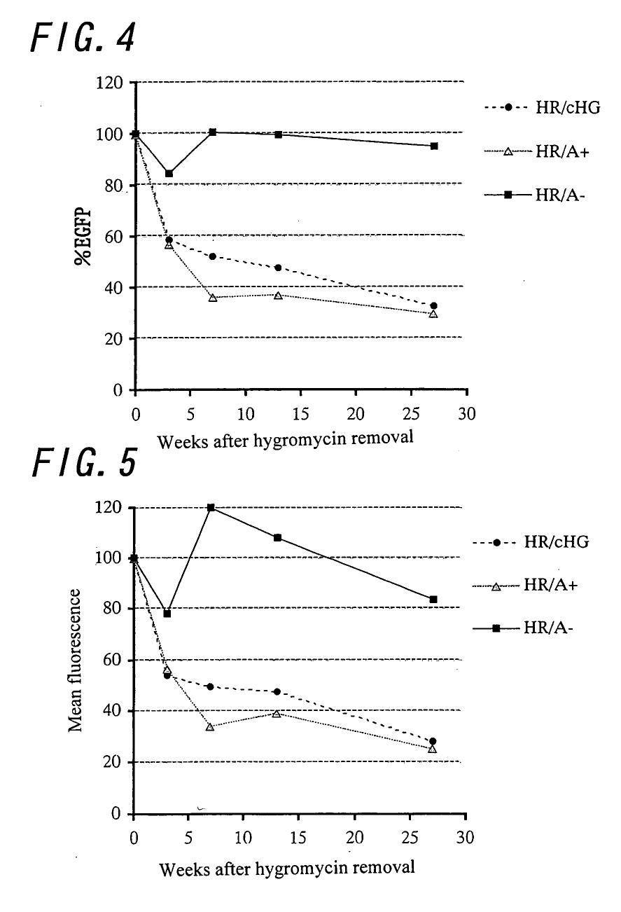

FIG. 3 illustrates histograms of fluorescence activated cell sorter (FACS) analysis for EGFP expression. Transduced HL-60 cells were selected for hygromycin positive cells for 4 weeks, and cultured extensively without hygromycin. Histograms in FIG. 3 show data from the 27th week after hygromycin removal. In FIG. 3, line a indicates untransduced cells, line b indicates cells cultured under hygromycin selection and filled histogram c indicates cells cultured in the absence of hygromycin. Sequential changes of EGFP expression after removal of hygromycin are shown by percentage of EGFP positive cells (FIG. 4) and by mean fluorescence intensity (FIG. 5).

In HL-60 cells, EGFP expression from HR/cHG (filled circle) and HR/A+(open triangle) started to diminish soon after removal of hygromycin, and the rates of EGFP positive cells and mean fluorescence intensity (MIFF) values declined to approximately 30% of their hygromycin selected counterparts (FIGS. 4 and 5). Alternatively, EGFP expression from HR/A− (filled square) was maintained throughout the assay period (FIGS. 4 and 5). This orientation-specific activity of ArsI is consistent with a previous report in which only the anti-sense orientation blocked the enhancer-promoter interaction in sea urchin and protected the transgene from position effect in Heal cells.

(Comparison of ArsI and cHS4 in their Anti-Silencing Capacity.)

To compare ArsI and cHS4 in their anti-silencing capacity, the inventors utilized a moloney-murine leukemia virus (MoMLV)-based vector and inserted insulator fragments into 3′-U3. RAVE/A+ and RAVE/A− are the MoMLV-based vectors containing ArsI in sense and anti-sense orientation, respectively. RAVE/H+ and RAVE/H− are the MoMLV-based vectors containing cHS4 insulator in sense and anti-sense orientation, respectively. HG/HG is control MoMLV-based vector without any insulator.

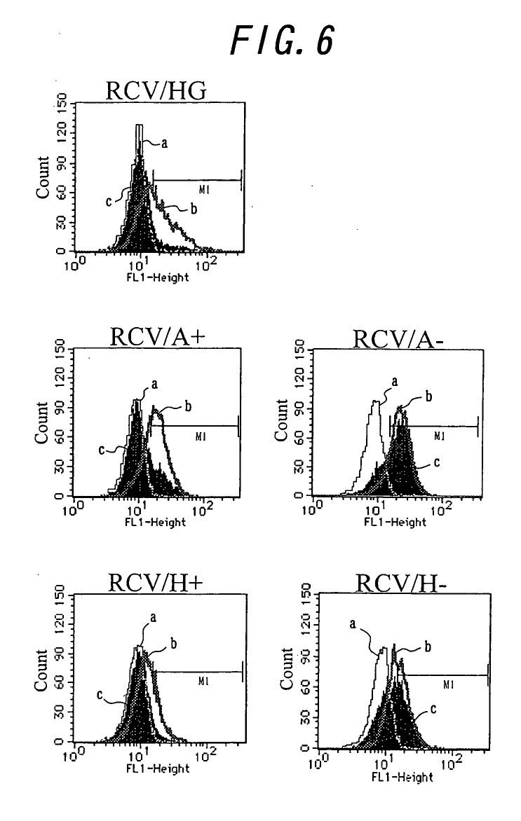

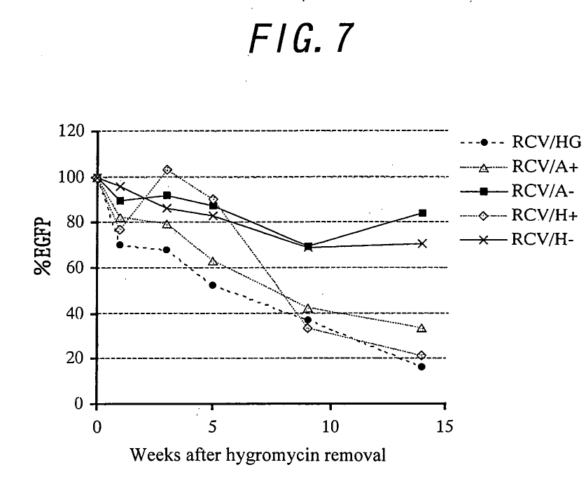

In FIG. 6, comparison of anti-silencing capacity between ArsI and cHS4 is shown. MoMLV-based vector (HG) was introduced into HL-60 cells. HL-60 cells were transduced with HG containing ArsI or cHS4, and then their EGFP expressions were monitored periodically. FIG. 6 are histograms of EGFP expression 14 weeks after hygromycin removal. In FIG. 6, line a indicates untransduced cells, line b indicates cells cultured under hygromycin selection and filled histogram indicates cells cultured in the absence of hygromycin. FIG. 7 shows time course of EGFP expressions.

Both ArsI and cHS4 were copied to 5′-U3 and remained intact after integration. HG vectors with or without insulators were introduced into HL-60 cells and their EGFP expressions were traced. EGFP expression from control vector without insulator (HG/HG) (filled circle) extinguished within the assay period rather faster than with the HIV-based vector (Oentiviral vector) (FIG. 4 and 7). A/A+ (open triangle) was also silenced, while A/A− (filled square) was protected from silencing (FIG. 7). Similar orientation dependency was also observed in cHS4-containing vectors (A/H+, A/H−) and their anti-silencing capability of cHS4 was equivalent to that of ArsI in HL-60 cells.

(Effect of ARS Insulator on Position Effect Variegation in HL-60 Cells.)

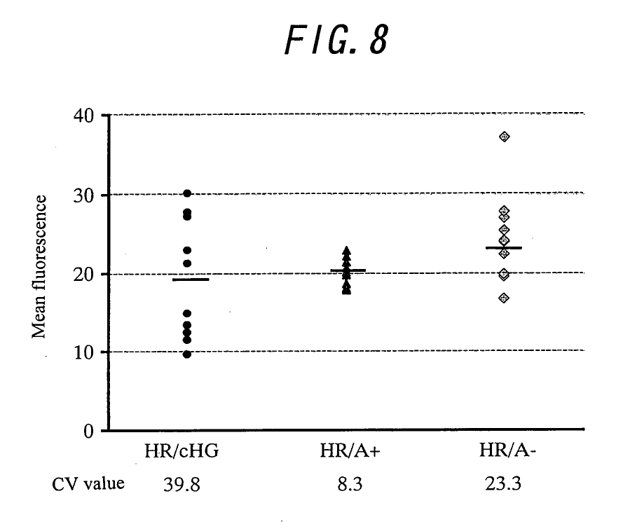

The inventors next tested the effect of ArsI on position effect variegation because assurance of position independent expression by an insulator may play a significant role in protection against silencing. HL-60 cells were transduced with HR/cHG, HR/A+ or HR/A− and selected for hygromycin resistant cells, and subsequently cloned by limiting dilution. Ten clones were selected from each cell pool, and tested for their EGFP expression. FIG. 8 shows the result of clonal analysis of transduced HL-60 cells. Clones were selected by limiting dilution of hygromycin-resistant cell pools and were maintained in the presence of hygromycin. Dot indicates the mean fluorescence intensity value of each clone. Co efficiency of variance (CV) is shown under the vector name.

Mean fluorescence intensity (MIFF) values of HR/cHG clones varied from 9.6 to 30.1 with CV of 39.8 (FIG. 8). Surprisingly, both HR/A+ and HR/A− clones showed unified expression. HR/A+ clones showed more unified expression than HR/A− clones with CV of 8.3 and 23.3 respectively (FIG. 8). These results indicated that ArsI protects lentiviral vectors from position effect variegation regardless of the orientation, suggesting that this activity is independent of silencing protection.

According to the present invention, a method to protect a transgene from silencing, wherein an insulator from sea urchin arylsulfatase gene is introduced concurrently with the transgene, is provided. Furthermore, according to this invention, a method for introducing of a gene, wherein an insulator from sea urchin arylsulfatase gene is introduced concurrently with a transgene so as to protect a transgene from silencing, is provided. Furthermore, according to this invention, a method for production of a vector, wherein an insulator from sea urchin arylsulfatase gene is introduced into the vector so as to protect the vector from silencing, is provided. Because the viral vector containing an insulator from sea urchin arylsulfatase gene can protect from silencing, a novel method useful for gene therapy is provided.

1. A method to protect a transgene from silencing, wherein an insulator from sea urchin arylsulfatase gene is introduced concurrently with the transgene.

2. The method to protect a transgene from silencing according to claim 1, wherein the transgene is introduced using a viral vector.

3. The method to protect a transgene from silencing according to claim 2, wherein said viral vector is a lentiviral vector or a retroviral vector.

4. The method to protect a transgene from silencing according to claim 1, wherein said insulator from sea urchin arylsulfatase gene is introduced in anti-sense orientation.

5. A method for introducing a gene, wherein an insulator from sea urchin arylsulfatase gene is introduced concurrently with a transgene so as to protect the transgene from silencing.

6. The method for introducing a gene according to claim 5, wherein gene transfer is performed using a viral vector.

7. The method for introducing a gene according to claim 6, wherein said viral vector is a lentiviral vector or a retroviral vector.

8. The method for introducing a gene according to claim 5, wherein said insulator from sea urchin arylsulfatase gene is introduced in anti-sense orientation.

9. A method for production of a vector, wherein an insulator from sea urchin arylsulfatase gene is introduced into the vector so as to protect the vector from silencing.

10. The method for production of a vector according to claim 9, wherein said vector is a viral vector.

11. The method for production of a vector according to claim 9 or claim 10, wherein said vector is a lentiviral vector or a retroviral vector.

12. The method for production of a vector according to claim 9, wherein said insulator from sea urchin arylsulfatase gene is introduced in anti-sense orientation.

13. A vector for introducing a transgene comprising an insulator from sea urchin arylsulfatase gene so as to protect the transgene from silencing.

14. The vector according to claim 13, wherein said vector is a viral vector.

15. The vector according to claim 13, wherein said vector is a lentiviral vector or a retroviral vector.

16. The vector according to any of claim 13, wherein said insulator from sea urchin arylsulfatase gene is introduced in anti-sense orientation.

17. The method to protect a transgene from silencing according to claim 2, wherein said insulator from sea urchin arylsulfatase gene is introduced in anti-sense orientation.

18. The method to protect a transgene from silencing according to claim 3, wherein said insulator from sea urchin arylsulfatase gene is introduced in anti-sense orientation.

19. The method for introducing a gene according to claim 6, wherein said insulator from sea urchin arylsulfatase gene is introduced in anti-sense orientation.

20. The method for introducing a gene according to claim 7, wherein said insulator from sea urchin arylsulfatase gene is introduced in anti-sense orientation.

21. The method for production of a vector according to claim 10, wherein said vector is a lentiviral vector or a retroviral vector.

22. The method for production of a vector according to claim 10, wherein said insulator from sea urchin arylsulfatase gene is introduced in anti-sense orientation.

23. The method for production of a vector according to claim 11, wherein said insulator from sea urchin arylsulfatase gene is introduced in anti-sense orientation.

24. The vector according to claim 14, wherein said vector is a lentiviral vector or a retroviral vector.

25. The vector according to claim 14, wherein said insulator from sea urchin arylsulfatase gene is introduced in anti-sense orientation.

26. The vector according to claim 15, wherein said insulator from sea urchin arylsulfatase gene is introduced in anti-sense orientation.
